(19) United States
(12) Patent Application Publication
(10) Pub. No.: US 2024/0003872 A1
(43) Pub. Date: Jan. 4, 2024

(54) METHODS FOR ASSAYING AND MEASURING THE EFFICACY OF ANTI-NAUSEA COMPOUNDS

(71) Applicant: CASTOR, Trevor, p ., Wobum, MA (US)

(72) Inventor: Trevor Percival Castor, Arlington, MA (US)

(73) Assignee: Aphios Corporation, Arlington, MA (US)

(21) Appl. No.: 18/037,028

(22) PCT Filed: Nov. 15, 2021
(86) PCT No.: PCT/US21/59429

§ 371 Date: May 15, 2023

Related U.S. Application Data
(60) Provisional application No. 63/113,939, filed on Nov. 15, 2020.

(51) Int. Cl. G01N 33/50

(52) U.S. Cl. CPC G01N 33/5061

(57) ABSTRACT

This invention is for methods to quantify the inhibition of 5-HT induced contraction of human intestinal smooth muscle and an assay to measure the effects of compounds on nausea, irritable bowel syndrome and irritable bowel disease. This invention can be used to measure the incidence and severity of nausea, and can be used as quality control assay for developing and releasing medications for controlling nausea and emesis.

### GOVERNMENT SUPPORT

Embodiments of the present invention were conceived and reduced to practice without Federal sponsorship or funding.

### FIELD OF THE INVENTION

This invention relates to methods for measuring the inhibition of 5-HT induced contraction of human intestinal smooth muscle and assaying the effects of compounds on nausea, irritable bowel syndrome and irritable bowel disease.

### REFERENCES TO OTHER PATENTS

This application discloses improvements and enhancements to methods and compositions of gingerols in U.S. Pat. No. 8,435,575 to Castor which are hereby incorporated by reference in its entirety.

This application discloses improvements and enhancements to methods and compositions of gingerols in PCT Patent Application No. US2019/064744 to Castor which are hereby incorporated by reference in its entirety.

This application is being filed simultaneously on the same date with related inventions as disclosed in U.S. Provisional Patent Application No. 63/113,941 to Castor, which are hereby incorporated by reference in their entirety.

### BACKGROUND OF THE INVENTION

Despite the widespread use of the 5-HT₃ receptor antagonist antiemetics, ondansetron (Zofran®, Glaxo Wellcome Oncology/HIV, Research Triangle Park, NC) in 1991, granisetron (Kytril,® SmithKline Beecham Pharmaceuticals, Philadelphia, PA) in 1994, and dolasetron mesylate (Anzemet,® Hoechst Marion Roussel, Kansas City, MO), post-chemotherapy nausea and vomiting continue to be reported by up to 70% of patients receiving highly emetogenic chemotherapy agents, such as cisplatin, carboplatin and doxorubicin. Research also suggests that the 5-HT₃ receptor antagonists are clinically more effective against emesis than they are against nausea.

Delayed post-chemotherapy nausea is a particularly difficult problem as it does not develop until after the patient has left the treatment location and is not well-controlled by currently available antiemetics. Data from a recently completed URCC CCOP Research Base study of patients receiving cisplatin, carboplatin or doxorubicin indicates that although nausea from receipt of these drugs is most likely to develop within the first 48 hours after administration of chemotherapy, in 18% of the patients it was first reported on or after Day 3 of the cycle.

Patients who suffer from post-chemotherapy nausea may also develop symptoms in anticipation of treatment. Anticipatory nausea (AN) is reported by approximately 20% of patients at any one chemotherapy cycle and by 25-30% of patients by the fourth chemotherapy cycle. Anticipatory vomiting (AV) develops in 8-20% of patients. No pharmacologic agents have had success in treating AN once it has occurred, and, although the behavioral method of systematic desensitization can be effective, it is not readily available in most clinic settings.

All in all, there is still a great deal of room for improvement in the control of nausea and vomiting (NV) associated with chemotherapy for cancer. Furthermore, antiemetics currently in widespread use have been associated with significant adverse effects, such as sedation, extra-pyramidal side effects and hypotension (associated with dopamine antagonists), as well as headache, diarrhea or constipation (associated with 5-HT₃ receptor antagonists). A desirable attribute in any substitute or additional antiemetic medication would be the absence of clinically significant adverse effects.

Ginger, an ancient spice mentioned in both the Bible and the Koran, is most known for its role as a flavoring agent for food in Asian and Indian recipes. Since the 16^{th} century, the dried aromatic rhizome (underground stem) of ginger (Zingiber Officinale, Roscoe), has also been used by practitioners of both Indian (Ayurvedic) and traditional Chinese medicine to treat gastrointestinal upsets such as nausea and excessive flatulence. North American folklore also recognizes the ability of ginger to relieve gastrointestinal upsets including nausea. Ginger is also believed to be the only herb that can prevent symptoms of motion sickness and it has been approved for that use by Germany's Commission E, the agency responsible for regulating the use of herbal products in that country. Recently ginger has been studied scientifically for its effect on nausea and vomiting associated with motion sickness, surgery and pregnancy.

In an early randomized trial, ginger was more effective than diphenhydramine (Dramamine®) and each was more effective than dried chickweed herb placebo in preventing gastrointestinal symptoms caused by vection induced motion sickness in a study of college students with self-reported high susceptibility to motion sickness. Ginger was also more effective than placebo in reducing vomiting related to seasickness in a group of naval cadets. Fewer episodes of nausea were also reported by the 40 cadets who received the ginger although the difference was not statistically significant. When used to prevent motion sickness, it is frequently suggested that the ginger be started one to two days before the trip and be continued throughout the period of travel.

A number of published studies have addressed the use of ginger for prevention of post-operative nausea and vomiting although the results have been mixed. Two studies comparing ginger (0.5 gm or 1 gm) vs. metochlopramide (10 mg) vs. placebo for control of post-operative nausea in women undergoing gynecologic surgery demonstrated equal effectiveness of ginger and metochlopramide for post-operative nausea; in both studies ginger and metochlopramide were significantly more effective than placebo. Phillips and co-investigators reported no significant differences in frequency of emesis between the three arms while Bone and colleagues reported less vomiting for both active drugs than for placebo. In the study headed by Phillips, participants assigned to the ginger arm required significantly less post-operative “rescue” antiemetic treatment.

Two other studies of post-operative nausea and vomiting found no significant effect of ginger. In one of these, 0.5 gm or 1.0 gm of ginger given pre-operatively had no greater effect than placebo on the frequency or severity of nausea or the frequency of vomiting. Outcomes were assessed three hours post-operatively. However, no standard antiemetic arm was included in the study design. Visalyaputra and colleagues compared 2.0 gm of oral ginger, 1.25 mg intravenous droperidol and placebo in a randomized fashion and reported no differences in the frequency or severity of post-operative nausea or the frequency of post-operative vomiting during the 24 hour period immediately following surgery. Potential confounding factors in studies of post-operative nausea and vomiting include the nausea inducing effect of agents used to induce and maintain anesthesia and provide pain relief and short assessment periods, allowing little time for ginger to exert its maximum anti-nausea effects.

A recent study of ginger for nausea and vomiting of pregnancy found a significant reduction of nausea over four days of treatment in women who were experiencing either nausea or vomiting. Sixty women were randomized in equal proportions to receive 250 mg of dried ginger or placebo in identical-appearing capsules four times daily for four days (a total of 1 gm of ginger each day). By the fourth day, nausea scores were significantly lower in the group of 32 women taking ginger than in the 35 women in the placebo arm. In an earlier randomized, controlled cross-over study, thirty women with hyperemesis gravidarum also reported that ginger was more effective than placebo over a four-day period.

Remarkably little published work has addressed the efficacy of ginger for prevention or treatment of nausea and vomiting caused by receipt of chemotherapy for cancer, and those studies that are available are plagued by design inadequacies including small sample sizes and non-validated assessment methods. In a published nursing doctoral dissertation, Pace studied 20 patients being treated for leukemia with cytosine arabinoside (ARA-C). Participants were given 10 mg intravenous Compazine® (prochlorperazine) prior to chemotherapy and every four hours for nine additional doses. They were also randomly assigned to receive either ginger capsules (0.5 g prior to chemotherapy followed by nine additional doses four hours apart) or an identical-appearing placebo on the same schedule. Participants who received ginger had significantly less severe nausea on the day of chemotherapy and on the following day than those taking the placebo capsules. Another study compared ginger (1.5 gm) to psoralen in patients receiving 8-MOP for extra-corporeal chemotherapy and found that the total nausea score was reduced by approximately one-third in those receiving ginger. Table 1 on the following page summarizes the results of previously conducted controlled studies of ginger for nausea.

TABLE 1
Studies Examining the Antiemetic Efficacy of Ginger.

| References | Sample | Study design | Treatment | Results |
|---|---|---|---|---|
| Grontved, | N = 80 | Two-arm, randomized, | 1.0 g ginger powder | Ginger was more effective |
| et al. (1988) | cadets | placebo-controlled trial for | or placebo given once | than placebo in controlling |
| | | control of seasickness. | | vomiting and cold sweats, |
| | | | | P < .05, but not for vertigo |
| | | | | or nausea. |
| Fischer- | N = 60 | Placebo-controlled, | 250 mg dried ginger | Greater relief with ginger |
| Ramussen, | Pregnant | randomized, crossover study | capsules or placebo | than placebo, P < .05. |
| et al. (1991) | women with | with 2-day washout, for | 4× daily for 4 days | |
| | severe nausea | nausea and vomiting | | |
| | | associated with morning | | |
| | | sickness. | | |
| Bone, | N = 60 | Three-arm, randomized, | Arm 1 = 1.0 g ginger | Ginger was more effective |
| et al. (1990) | Women | placebo-controlled trial of | Arm 2 = 10 mg | than placebo, P < .05 and |
| | undergoing | ginger and metochlopramide | metochlopramide | similar in effectiveness to |
| | gynecological | for control of post-operative | Arm 3 = placebo | metochlopramide for |
| | surgery | nausea and vomiting. Study | | nausea and vomiting |
| | | medication was given orally | | control. |
| | | prior to surgery. | | |
| Pace 1986) | N = 41 | Two-arm, randomized, | 500 mg ginger or | Patients receiving ginger |
| | Patients | placebo-controlled trial of | placebo prior to | had less nausea than those |
| | receiving | Compazine ®, with or | treatment and every | receiving placebo. |
| | chemo- | without ginger for control of | four hours following | |
| | therapy | chemotherapy-induced | treatment for 36 hours | |
| | | nausea and vomiting. | | |
| Phillips, | N = 120 | 3 parallel arms, placebo | Arm 1 = 1 gram of | Less patients need |
| et al. (1993) | women after | controlled, either given | ginger powder given | antiemetics afterward |
| | lapara-scopic | metochlopramide or ginger | one hour prior to | with ginger, less |
| | gynaecological | for post-operative vomiting | anaesthesia | likely to be nauseated. |
| | surgery | and nausea | Arm 2 = metochlopramide | 21% nauseated with |
| | | | one hour prior to | ginger, 41% with |
| | | | anaesthesia | placebo, 27% |
| | | | | metochlopramide |
| Arfeen, | N = 108 | 3 parallel arms, placebo | Arm 1 = 0.5 g ginger 1 | The ginger did not appear |
| et al. (1995) | women after | controlled, given placebo or | hour prior to surgery | to have a statistically |
| | lapara-scopic | one of two doses of ginger | and 2 doses after | significant effect on the |
| | gynaecological | for reduction of post-surgical | Arm 2 = 1.0 g ginger | amount of nausea or |
| | surgery | nausea and vomiting | prior to surgery and 2 | vomiting post-surgery |
| | | | doses after | |
| | | | Arm 3 = placebo | |
| Visalyaputra | N = 120 | 4 parallel arms, placebo | Arm 1 = 1.0 ginger | Neither drug showed a |
| et al. (1998) | women after | controlled received either | presurgically | statistically significant |
| | lapara-scopic | ginger or 1.25 mg droperidol, | Arm 2 = 1.0 ginger | decrease in nausea or |
| | gynaecological | or both to reduce post- | and 1.25 droperidol | vomiting |
| | surgery | surgical nausea and vomiting | Arm 3 = 1.25 mg | |
| | | | droperidol | |
| | | | Arm 4 = placebo | |
| Meyer & Schwartz, | N = 11 | Single arm given psoralen at | 3 capsules of 530 mg | Those taking the ginger |
| et al. (1995) | patients | 0.5-0.6 mg/kg prior to | each administered 30 | reduced nausea by ⅓ |
| | undergoing 8-MOP | treatment, then evaluated, | min prior to 8-MOP | degree of level without |
| | treatments | next treatment given ginger | treatment | ginger |
| | | and evaluated | | |
| Mowrey & Clayson | N = 36 | 3 arm-randomized, placebo | Arm 1 = 100 mg | Ginger was found to be |
| (1982) | paid college | controlled, for vection | Dramamine ® | more effective at reducing |
| | students with | induced motion sickness | Arm 2 = 940 mg | vection induced motion |
| | self-rated | ginger versus placebo versus | ginger root | sickness than placebo as |
| | high | Dramamine ® | Arm 3 = placebo | measured by time in |
| | susceptibility | | given 25-30 minutes | induced motion. P < .001 |
| | to motion | | prior to test | |
| | sickness | | | |
| Vutyavanich | N = 70 | 2 arm, placebo controlled, | Arm 1 = 1.0 g ginger | Visual analog scores |
| et al. (2001) | pregnant | double-masked, randomized | for four days | decreased with ginger |
| | women at or | ginger versus placebo, | Arm 2 = placebo | p = .014, vomiting episodes |
| | prior to 17 | participants self-reported | | decrease with p < .001 |
| | weeks | nausea, vomiting, and | | And the improvement of |
| | gestation | severity. Lasted four days, | | those in ginger group with |
| | | and pre/post evaluation of | | p < .001 |
| | | symptoms | | |

Previous research suggests that ginger may be effective against nausea associated with chemotherapy, but design inadequacies and small numbers limit the power and generalizability of the results and no dose-response studies have been reported to date. A phase II/III randomized, dose-finding, placebo-controlled clinical trial was conducted to assess the efficacy of ginger (Zingiber Officinale) for nausea associated with chemotherapy for cancer in the CCOP member sites affiliated with the URCC CCOP Research Base. Innovative aspects of the study design include collecting baseline data on nausea following the second cycle of chemotherapy, beginning the intervention three days prior to chemotherapy to maximize the post-chemotherapy effect of ginger, assessing symptoms prior to taking any ginger, after three days of ginger alone, and after three days of ginger plus standard antiemetics at cycles three and four, assessing anticipatory nausea as well as acute and delayed post-chemotherapy nausea and using validated measures for outcome assessment. The primary outcome was assessment of nausea following one chemotherapy cycle with the intervention (the third cycle). The intervention was continued for the fourth cycle of chemotherapy to assess the consistency of any effectiveness of ginger for nausea as a secondary, exploratory analysis.

Ginger (Zingiber officinale) has been used for centuries as traditional medicine to treat nausea and vomiting; in vitro studies suggest that ginger compounds also have anti-cancer, anti-inflammatory and anti-spasmodic effects. Clinical trials indicate that ginger constituents reduce pregnancy and post-surgical nausea, vomiting and motion sickness.

Zindol®, an improved SuperFluids ginger extract, reduced acute nausea severity without significant side-effects or drug interactions in a phase 2/3 clinical trial. 5-HT3 receptor antagonists like Palonosetron (Aloxi®; Eisai), Ondansetron (Zofran®; GSK), Granisetron (Kytril®; Roche), and Dolasetron (Anzemet®; Aventis) are highly effective in limiting vomiting but have little or no impact on nausea.

Mechanisms underlying anti-emetic effects of ginger are not well known but the ginger phytochemicals, 6-gingerol, 8-gingerol, 10-gingerol, and 6-shogaol, may act as antagonists of 5-hydroxytryptamine (5-HT), NK1 and histamine receptors.

In the present study, the refined ginger extract, Zindol® (containing 6-, 8- and 10-gingerol (6-G, 8-G, 10-G) and 6-shogaol (6-S)) was evaluated for its ability to block 5-HT induced isotonic contractions of human intestinal smooth muscle cells (HISMCs) cultured in collagen-matrices. 5-HT at 10⁻⁷M, 10⁻⁸M and 10⁻⁹M induced dose and time dependent contraction of HISMC gels. Subsequent studies used 5-HT at 10⁻⁷M. HISMC tonic contractions towards 5-HT (10⁻⁷M) were reduced by Zindol (10⁻⁴M, 10⁻⁵M 10⁻⁶M and 10⁻⁷M) or Ondansetron (a selective 5-HT3 antagonist) in a time and concentration-dependent manner.

Because viability studies show that Zindol® does not appear to be toxic up to a dose of 10⁻⁴M, these data indicate that based on the antispasmodic effects of Zindolt in HISMC contraction studies, ginger constituents may represent a novel group of 5-HT receptor antagonists of intestinal smooth muscle contraction in the treatment of IBS and IBD-like disturbances.

### SUMMARY OF THE INVENTION

Embodiments of the present invention are directed to methods for the inhibition of 5-HT induced contraction of human intestinal smooth muscle and assay for effects of compounds on nausea, irritable bowel syndrome and irritable bowel disease.

### BRIEF DESCRIPTION OF THE DRAWINGS

FIG. 1 depicts contraction of HISMC Stimulated by 5-HT;

FIG. 2 shows effects of Ondansetron on 5-HT induced HISMC/Collagen Matrix Contraction;

FIG. 3 shows effects of Ondansetron on HISMC/collagen Matrix Contraction;

FIG. 4 shows effects of Zindol® on 5-HT induced HISMC/collagen gel contraction;

FIG. 5 shows effects of Zindol® on HISMC/collagen gel contraction;

FIG. 6 shows effect of Zindol, Z-oil, and DMSO on Cell Viability on Human Intestinal Smooth Muscle Cells (HISMCs);

FIG. 7 shows effect of Zindol, Z-oil, and DMSO on Cell Viability on Bovine Turbinate Cells (BTCs); and

FIG. 8 shows the effects of Emend (Aprepitant) on HISMC/collagen Matrix Contraction.

### DETAILED DESCRIPTION OF PREFERRED EMBODIMENTS

Materials utilized include human intestinal smooth muscle cells (HISMC) (ScienCell), rat-tail collagen type-1, smooth muscle cell medium (SMCM) (ScienCell), 1% smooth muscle cell growth supplement (SMCGS) (ScienCell), 2% fetal bovine serum (FBS), 1% penicillin/streptomycin (P/S) (ScienCell), 5-Hydroxytryptamine hydrochloride (Sigma), Ondansetron HCl (Sigma), poly-L-lysine stock solution (10 mg/ml, ScienCell, Cat. No. 0413), trypsin/EDTA solution (T/E), T-75 Flasks, cell culture incubator setting at 37° C. with 5% CO₂, Laminar flow hood, Eppendorf pipettes of different volumes, 12-, 24- and 96-well plates from Falcon, Phase contrast microscope, Epson scanner, and NIH Image J Imaging analysis program.

Experiments were performed to evaluate the contractile responsiveness of the human intestinal smooth muscle cells (HISMCs) in response to stimuli that induce or limit nausea and emesis.

Zindol® was obtained from Aphios Corporation (Woburn, MA). Human intestinal smooth muscle cells (HISMC), smooth muscle cell medium (SMCM), smooth muscle cell growth supplement (SMCGS), penicillin/streptomycin, and (P/S) poly-L-lysine were purchased from ScienCell (Carlsbad, CA). 5-Hydroxytryptamine hydrochloride (5-HT3), ondansetron hydrochloride, and Trypsin/EDTA solution (T/E) were purchased from Sigma. Rat-tail collagen type-1 and dialyzed fetal bovine serum (FBS) were a gift from Dr. Alexander (Shreveport, LU). Aprepitant (Emend®) was purchased from ADOOQ BioScience (Irvine, CA). Bovine turbinate cells (BTCs) were obtained from Aphios Corporation (Woburn, MA). Cytotoxicity assay, CellTiter 96® AQueous One Solution Reagent was purchased from Promega (Madison, WI).

### Cell Culture

HISMC cells were grown in poly-L-lysine coated T-75 flasks with SMCM supplemented with 2% dialyzed FBS, 1% of smooth muscle cell growth supplement (SMCGS), and 1% of penicillin/streptomycin antibiotics in a 5% CO₂ incubator. After HISMCs reached confluency, cells were trypsinzed at 37° C. for 2 minutes, harvested by centrifugation (1,000×rpm, Beckman Coulter centrifuge for 3 min), resuspended in fresh SMCM and subcultured to determine cell density for further experiments. Bovine turbinate cells (BTCs) were grown in DMEM supplemented with 10% FBS, and 1% of penicillin/streptomycin antibiotics in a 5% CO₂ incubator.

Zindol® is an enhanced SuperFluids extract of ginger formulated in Z-oil under cGMP for oral administration. Zindol® is standardized to different gingerols and 6-shogaol (Table 2). Gingerols are the major phenolic components of ginger and shogaols are the dehydrated forms of gingerols.

TABLE 2
Major Bioactive Components of Zindol ®

| Components | MW (g/mol) |
|---|---|
| 6-Gingerol (APH-02031) | 294.38 |
| 8-Gingerol (APH-02032) | 322.44 |
| 10-Gingerol (APH-02033) | 350.49 |
| 6-Shogaol (APH-02034) | 276.37 |
| Zindol ® Mean | 310.92 |

Each capsule of Zindol® contains a specific amount of gingerols and shogaol in a Z-oil vehicle. Z-oil consist of a mixture of vitamin E, Extra Virgin olive oil, lecithin, and medium chain triglycerides. Based on the concentration of Zindol® in a capsule, the molarity of Zindol® was calculated based on the average molecular weight of four bioactive compounds listed in the table on the right. The calculated molarity of Zindol® is 1.37×10⁻¹M.

Zindol® is hydrophobic and dissolves readily in Dimethyl sulfoxide (DMSO). DMSO was used to dissolve the formulated ginger extract to conduct in vitro studies. Zindol® and Z-oil vehicle were first dissolved in DMSO and diluted in SMCM solution without FBS and SMCGS. The concentration range of Zindol® and Z-oil was 10⁻⁹M to 10⁻³M or 0.001 μM to 1,000 μM. DMSO was diluted in the same concentration range of Zindol® and vehicle.

### Cell Contraction Assay

This assay was used to study the ability of HISMCs to reorganize and contract in a collagen matrix. All procedures were done accordingly to protocols described by Dr. Alexander (5-HT3 dependent smooth muscle contraction assay, revised Aug. 30, 2012). The assay consisted in the preparation of collagen solution and in the polymerization and release of HISMCs/collagen matrices.

For ˜12 mL of collagen/HISMCs, 15.0 mg of rat tail collagen was dissolved in 3.2 mL of cold 0.012M HCL. The solution was placed for 30 min in a rocker machine for gently agitation to avoid introducing bubbles. Then, 0.8 mL of cold PBS was added to the tube, mixed well, and kept on ice. The pH of the collagen solution was quickly titrated with 0.5 M NaOH to 7.45, and monitored by pH strips. The final collagen concentration was 1.25 mg/mL. The neutralized cold collagen solution was mixed with 12 mL of cold SMCM/1.5% dialyzed FBS containing HISMCs at desire cell density (1×10⁵ cells/mL). 0.5 mL of collagen/HISMCs mixture was added per well in 24-well plates and was incubated for 1 h at 37° C., 5% CO2.

After collagen polymerization, 0.5 mL of culture medium with or without two-time concentration of drug treatments was added atop each collagen gel lattice. The polymerized collagen matrix containing HISMCs was released from the edges of the well with a sterilized heat-blunted Pasteur pipette tip to allow them to float in culture medium, and contraction occurred with or without agonist addition in the absence of external mechanical load. The collagen gel size change (contraction index) was measured by morphometric analysis at various times (0, 24, 48, and 72 h). The culture plates were analyzed in triplicate and monitored at 24 h intervals. At the end of each time point, the plates were scanned via an Epson flatbed scanner.

The effect of Zindol®, and Aprepitant on the proliferation of the HISMCs and bovine turbinate cells (BTCs) cell lines was determined by the tetrazolium compound 3-(4,5-dimethylthiazol-2-yl)-5-(3-carboxymethoxy-phenyl)-2-(4-sulfophenyl)-2-Htetrazolium contained in the CellTiter 96® AQueous One Solution Cell Proliferation Assay (MTS assay).

Briefly, the HISMCs and BTCs were plated in 96-well plate at a density of 3,000 cells/well in their respective medium. The cells were then treated with or without various concentrations of Zindol® (10⁻⁶M to 10⁻³M) and incubated at 37° C. in a 5% CO₂ environment for 24 h, 48 h and 72 h. After the designated time period, 100 μL of culture medium was removed from the monolayers of the 96-well plate with a multichannel pipette. Then 20 μL of CellTiter 96® AQueous One Solution Reagent was added to each well of the 96-well assay plate. 20 μL of same solution was also added to wells containing medium with no cells used as blanks. The plate was then incubated at 37° C. for 4 hours. The absorbance was measured at 490 nm using a Synergy HT 96-well Plate Reader (BIO-TEK). The mean OD value for blanks was subtracted from the OD values obtained for the individual wells. All assays were performed in quadruplicate and the data were plotted on Excel spread sheet to calculate the cell viability.

### EXAMPLES

### Example 1: Contraction of HISMC Stimulated by 5-HT

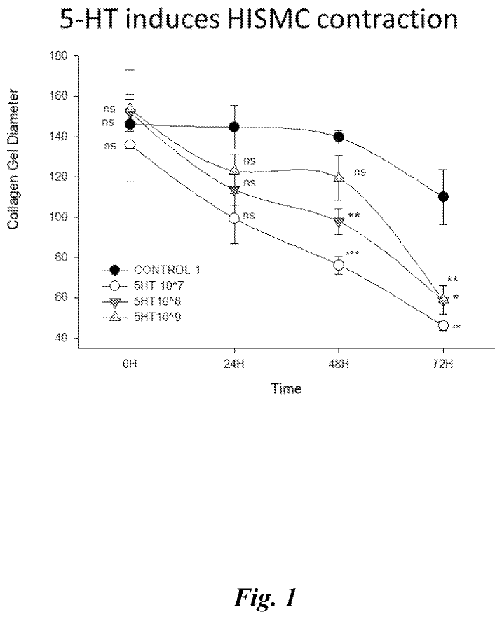

Contraction of HISMC/collagen gel was carried out for the times shown with 5-HT at different concentrations as shown in FIG. 1. Data are presented as means of triplicate wells per condition.

### Example 2. Effects of Ondansetron on HISMC/Collagen Matrix Contraction

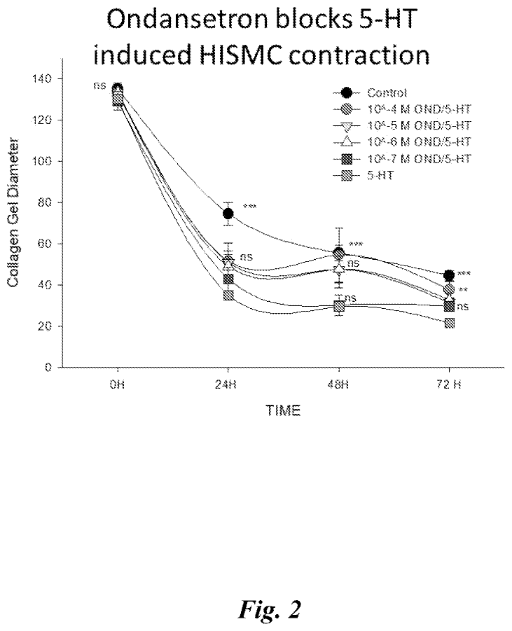

FIG. 2 shows the effects of Ondansetron on HISMC/Collagen matrix contraction. HISMC/collagen matrixes were incubated with conditioned medium alone or in the presence of different concentrations of Ondansetron with 5-HT. FIG. 2 shows Ondansetron inhibited HISMCs contractions in a concentration- and time-dependent manner. Data are presented as means of triplicate wells per condition.

### Example 3. Effects of Ondansetron on HISMC/Collagen Matrix Contraction

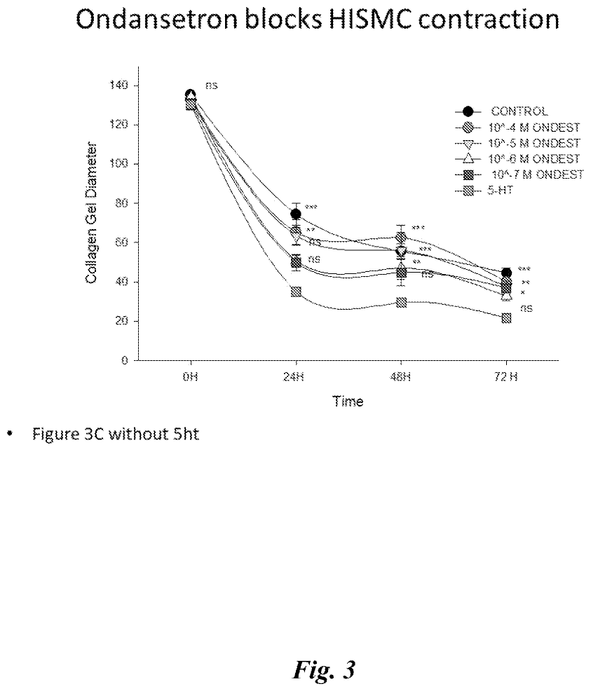

FIG. 3 shows the effects of Ondansetron on HISMC/Collagen matrix contraction. HISMC/collagen matrixes were incubated with conditioned medium alone or in the presence of different concentrations of Ondansetron without 5-HT. FIG. 2 shows Ondansetron inhibited HISMCs contractions in a concentration- and time-dependent manner. Data are presented as means of triplicate wells per condition.

### Example 4. Effects of Zindol® on HISMC/Collagen Gel Contraction

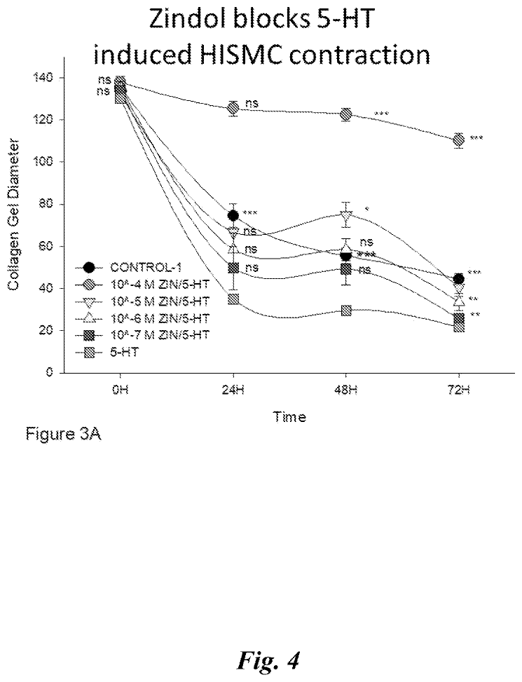

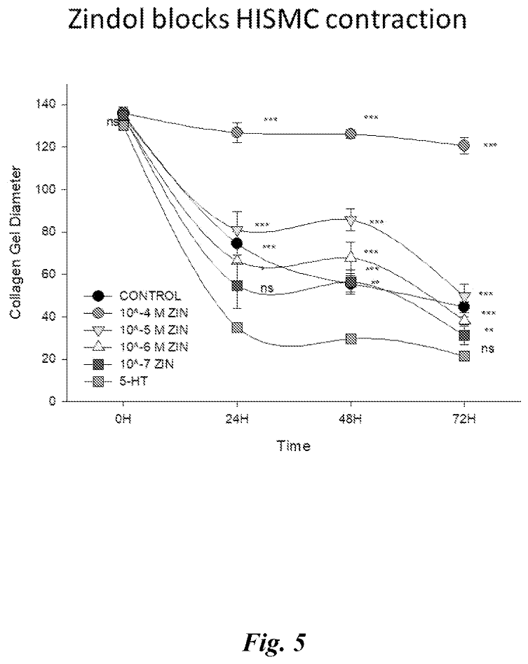

FIGS. 4 and 5 show the effects of Zindol® on HISMC/collagen gel contraction. HISMC/collagen gels incubated with conditioned medium alone or in the presence of different concentrations of Zindol® with 5-HT and without 5-HT, and 5-HT alone. FIGS. 4 and 5 show that Zindol® and attenuated collagen gel contraction in a concentration dependent manner. Data are presented as means of triplicate wells per condition.

### Example 5. Effect of Zindol, Z-Oil, and DMSO on Cell Viability in Human Intestinal Smooth Muscle Cells (HISMCs) and Bovine Turbinate Cells (BTCs)

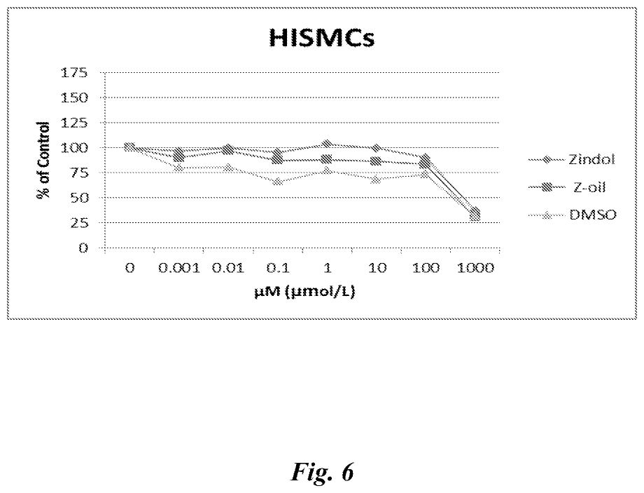

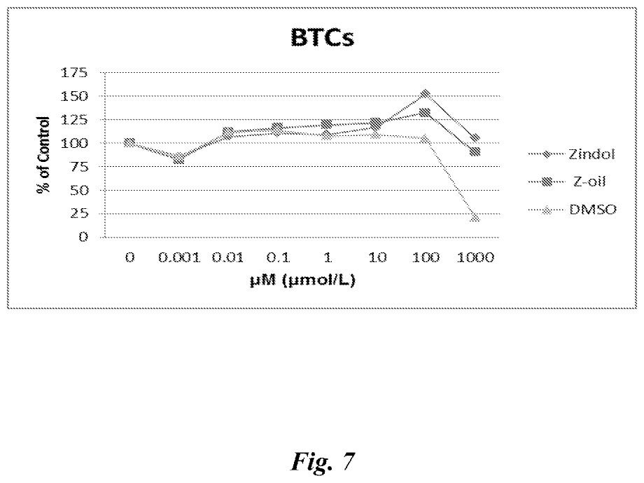

Cell proliferation assays, which measures metabolic levels, were performed at 24 h following drug treatment and the values are expressed as a percentage of the control wells. FIG. 6 shows that Zindol and Z-oil had no cytotoxic effects on smooth muscle cells (HISMCs) up to concentrations of 100 μM, and that cytotoxic effects at higher concentrations were probably caused by the DMSO solvent. FIG. 7 shows that Zindol and Z-oil had no cytotoxic effects on bovine turbinate cells (BTCs) up to concentrations of 100 μM, and that cytotoxic effects at higher concentrations were probably caused by the DMSO solvent. Data are presented as means of triplicate wells per condition.

### Example 6. Effects of Aprepitant in HISMC/Collagen Matrix Contraction

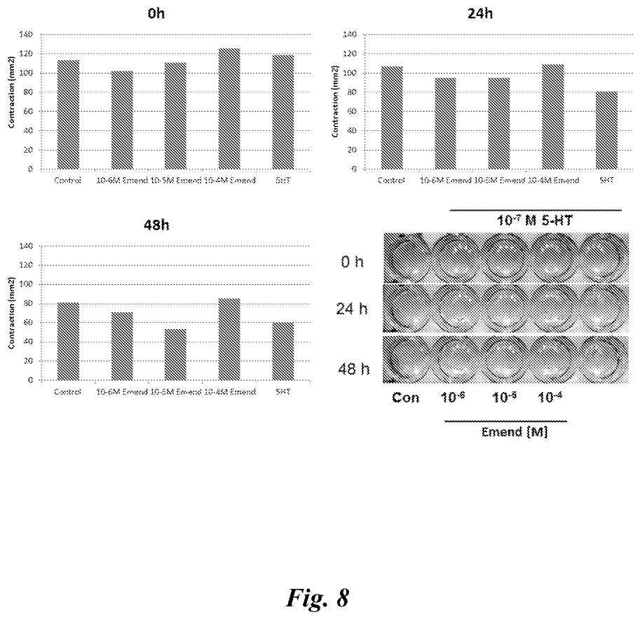

HISMC/collagen gels were incubated with conditioned medium alone or in the presence of different concentrations of Aprepitant (Emend) with or without 5-HT3. FIG. 8 shows that Aprepitant (Emend) attenuated collagen gel contraction only at high concentrations, 10⁻⁴ M and not significantly. Data are presented as means of triplicate wells per condition.

It is intended that the matter contained in the preceding description be interpreted in an illustrative rather than a limiting sense.

What is claimed is:

1. A method or assay for measuring the effectiveness of test compounds for blocking or inhibiting 5-HT induced contraction of human intestinal smooth muscle cells (HISMC), comprising the steps of:
(a) providing an in vitro sample of human intestinal smooth muscle cells (HISMC) in a cell culture growth medium for assay;
(b) providing a sample test compound, with 5-HT3 antagonist properties, which inhibits contraction of human intestinal smooth muscle cells ((HISMC);
(c) mixing or combining a selected amount of the test compound in a predetermined concentration with the in vitro sample of human intestinal smooth muscle cells ((HISMC);
(d) measuring the contractile responsiveness of the HISMC sample to the test compound by measuring cell contraction vs time for a predetermined test period;
(e) repeating the measuring using different concentrations of the test compound; and
(f) determining, based on the measured data, the efficacy of the test compound to inhibit 5-HT induced contractions of the human intestinal smooth muscle cells (HISMC).

2. The method of claim 1, further including the step of providing a control sample, wherein the human intestinal smooth muscle cells (HISMC) are left untreated.

3. The method of claim 1, wherein the human intestinal smooth muscle cells (HISMC) are cultured in collagen matrices in a gel formulation.

4. The method of claim 1, wherein the test compound includes gingerol or ginger-based compounds.

5. The method of claim 1, wherein the test compound includes Zindol.

6. The method of claim 5, wherein the test compound includes Z-oil.

7. The method of claim 1, wherein the test compound includes Ondansetron.

8. The method of claim 1, wherein the test compound includes Aprepitant.

9. The method of claim 1, wherein the test compound includes a DMSO solvent.

10. The method of claim 1, wherein the assay of performed with bovine turbinate cells (BTC).

11. The method of claim 1, wherein the efficacy of the test compound is determined for treating post-chemotherapy nausea.

12. The method of claim 1, wherein the efficacy of the test compound is determined for treating post-operative nausea.

13. The method of claim 1, wherein the efficacy of the test compound is determined for treating irritable bowel syndrome and irritable bowel disease.

14. A kit for measuring the effectiveness of test compounds for blocking or inhibiting 5-HT induced contraction of human intestinal smooth muscle cells (HISMC), comprising the steps of:
(g) providing an in vitro sample of human intestinal smooth muscle cells (HISMC) in a cell culture growth medium for assay;
(h) providing a sample test compound, with 5-HT3 antagonist properties, which inhibits contraction of human intestinal smooth muscle cells ((HISMC);
(i) mixing or combining a selected amount of the test compound in a predetermined concentration with the in vitro sample of human intestinal smooth muscle cells ((HISMC);
(j) measuring the contractile responsiveness of the HISMC sample to the test compound by measuring cell contraction vs time for a predetermined test period;
(k) repeating the measuring using different concentrations of the test compound; and
(l) determining, based on the measured data, the efficacy of the test compound to inhibit 5-HT induced contractions of the human intestinal smooth muscle cells (HISMC).
